(19) United States
(12) Patent Application Publication
(10) Pub. No.: US 2008/0138440 A1
(43) Pub. Date: Jun. 12, 2008

(54) Methods of diagnosing and alleviating gadolinium toxicity

(75) Inventors: Sundararaman Swaminathan, Little Rock, AR (US); Sudhir V. Shah, Little Rock, AR (US)

Correspondence Address: COVINGTON & BURLING, LLP;ATTN: PATENT DOCKETING, 1201 PENNSYLVANIA AVENUE, N.W., WASHINGTON, DC, 20004-2401, US

(73) Assignee: CorMedix Inc., Summit, NJ (US)

(21) Appl. No.: 11/987,166

(22) Filed: Nov. 28, 2007

Related U.S. Application Data
(60) Provisional application No. 60/861,440, filed on Nov. 29, 2006.

(51) Int. Cl. A61K 33/24; A61K 38/02; A61K 31/16; A61P 39/04; A61B 5/00

(52) U.S. Cl. 424/617; 514/2; 514/616; 600/300; 128/898

(57) ABSTRACT

The present invention relates to novel methods of treating or reducing the likelihood of developing gadolinium toxicity by administering to a patient a metal chelator before, concurrently with, or after exposure to gadolinium. The novel methods comprise, inter alia, the use of iron chelators to aid patients at increased risk of developing a gadolinium-induced condition, such as nephrogenic systemic fibrosis, acute kidney injury, cardiovascular disease and accelerated senescence.

### CROSS-REFERENCE TO RELATED APPLICATIONS

This application claims the benefit of U.S. Provisional Application No. 60/861,440, filed Nov. 29, 2006, the entire teachings of which are incorporated herein by reference.

### BACKGROUND OF THE INVENTION

Nephrogenic fibrosing dermopathy/nephrogenic systemic fibrosis (NFD/NSF) is a newly described cutaneous disorder in patients with renal insufficiency characterized by exaggerated wound healing response, the trigger for which is unknown. Specific histologic features may include mucin deposition, dermal and systemic infiltration of CD34⁺ (a stem cell marker) spindle cells, and presence of CD68+ multinucleated giant cells. Pathology may extend locally into subcutaneous tissues and muscles, and recently, systemic fibrosis of organs such as diaphragm, atrial myocardium, dura mater and testes have been described. Clinically, NFD/NSF may typically manifest itself with erythematosus plaques with edema and woody induration of extremities and trunk. Functional consequences of NSF are severe and may result in severe limitation of joint mobility and even death, as patients withdraw from dialysis secondary to poor quality of life and intolerable pain. Incidence of NSF is currently unknown and it is believed that, any estimate based on current reporting is likely to result in gross underestimation of the problem as the disease is not well-recognized. Described associations with NSF include high-dose erythropoietin (EPO) therapy and exposure to gadolinium chelates.

Association of gadolinium magnetic resonance contrast agents with NSF was only recently described. On prospective follow-up, end stage renal disease (ESRD) patients undergoing magnetic resonance angiogram with gadodiamide were reported to develop NSF within days to weeks of exposure. In these studies, however, many dialysis patients who were exposed to gadolinium contrast did not develop NSF and a few NSF patients did not manifest any clinical signs of NSF upon their previous exposure to gadodiamide.

The mechanism for the association of the development of NSF after administration of gadolinium is unknown. An initial report by Grobner et al. suggested that these patients were acidotic when they received gadolinium and this favored gadolinium's dissociation from its chelate that lead to its deposition in the tissues as gadolinium carbonate and phosphate. However, a subsequent report failed to show low venous bicarbonate at the time of gadolinium exposure. Recently, the United States Food and Drug Administration (FDA), recognizing that there are no known mechanisms for its toxicity, recommended hemodialysis for three consecutive days after exposure to remove gadolinium.

It is well known that free gadolinium (i.e., gadolinium disassociated from its chelate) is toxic and gadolinium is therefore routinely administered in chelated form. In clinical studies, administration of gadolinium has been associated with mild toxicity. In vitro and in vivo data indicate that gadolinium may be released from a chelator into free form via transmetallation exchange of copper, calcium, iron, or zinc cations in the chelate. Thus, the toxicity of gadolinium-containing compounds depends greatly upon the stability of gadolinium complexes in a given environment, and the presence of factors that may initiate the dissociation of gadolinium from these complexes.

It is noteworthy that magnetic resonance contrast agents are known to mobilize zinc and cause transient (up to 72 hours) elevations in serum iron in 15-30% of healthy volunteers. Because iron is tightly bound to ferritin and hemosiderin, it is thought that its free concentration is insufficient to make transmetallation reactions a concern.

### SUMMARY OF THE INVENTION

To help address concerns pertinent to the development of metal-associated gadolinium toxicity, a method of identifying a patient at risk for gadolinium toxicity was devised, comprising determining a clinical condition of the patient known to be associated with or predisposed to fibrosis, inflammation, or both; and correlating the clinical condition with risk for gadolinium toxicity.

Likewise, a method of the present invention can be used for identifying a patient at risk for gadolinium toxicity, the method comprising measuring an index of iron status selected from the group consisting of serum free iron, total iron-binding capacity of the patient, transferrin levels, transferrin saturation, bone marrow iron stores, and hepatic iron stores; and correlating the index of iron status with an elevated patient risk for gadolinium toxicity. An increase in transferrin saturation more than about 25% can render the patient at increased risk of the patient for gadolinium toxicity. The increase in transferrin saturation of the patient is determined relative to one of: a baseline value of transferrin saturation expected of a healthy patient, a baseline value of the patient, and a value of transferrin saturation for the patient measured prior to the administering. An increase of more than about 50%, or preferably more than about 25%, in serum iron of the patient can be associated with an increased risk of the patient for gadolinium toxicity. An increase in serum total iron-binding capacity of more than about 200 μg/dL, or more particularly more than about 150 μg/dL can be associated with an increased risk of the patient for gadolinium toxicity. An increase of more than 50% in serum ferritin, or preferably more than about 20%, or a serum ferritin value of greater than about 500 ng/mL, can be associated with an increased risk of the patient for gadolinium toxicity. An increase of more than about 50% in oxidative stress, or preferably more than about 25%, can be associated with increased risk of the patient for gadolinium toxicity. An increase of more than about 50%, or preferably more than about 25%, in catalytic iron can be associated with an increased risk of the patient for gadolinium toxicity. Similarly, a decrease of more than about 20% in the hepcidin level of a patient can be associated with an increased risk of the patient for gadolinium toxicity. As with transferrin saturation, with respect to each index of iron status, the change in that index is determined relative to a baseline. The baseline can be the value of the index expected of a healthy patient, a baseline value of the particular patient, or a value of the index measured for that patient prior to administering the gadolinium.

Because of the potential for serious health threats due to metal-associated gadolinium toxicity in humans, a method for treating, or at least reducing the likelihood of developing symptoms of, gadolinium toxicity in a human exposed to gadolinium was designed. The method comprises in certain embodiments administering a pharmaceutically effective amount of a metal chelator to the human that is capable of reducing or eliminating the symptoms of conditions associated with gadolinium exposure. The metal chelator may be administered in combination with gadolinium as described in this specification.

Numerous metal chelators are suitable for use with the invention. Examples of suitable metal chelators include ethylenediamine tetra-acetic acid, N-acetylcysteine, hydroxyquinoline, deferiprone, deferasirox, deferitrin, deferoxamine, polyanionic amines, substituted polyaza compounds, 2-pyridylcarboxyaldehyde isonicotinoyl hydrazones, di-2-pyridylketone isonicotinoyl hydrazones, di-2-pyridylketone thiosemicarbazones, and 3-aminopyridine-2-carboxaldehyde-thiosemicarbazone.

Aspects of the inventive methods are suited to prevent, slow or reduce the risk of gadolinium toxicity in a patient, or to treat a patient who has symptoms of gadolinium toxicity, which can manifest as conditions such as NSF, acute kidney injury, cardiovascular disease, accelerated senescence—including death—and high levels of gadolinium chelates in body tissues, such as skin, or body fluids including blood and plasma. Accelerated senescence means that usual aging processes occur at a faster than normal rate, and can manifest in gadolinium-exposed patients as one or more conditions such as vascular calcification, osteoporosis, skin wrinkling, dementia, and cancers such as basal cell and squamous cell skin cancers that are correlated with senescence.

The novel methods can also aid patients who have a pre-existing condition that can be exacerbated by gadolinium exposure. The pre-existing condition may be, for example, chronic kidney disease, myocardial fibrosis, multiple sclerosis, inflammation or systemic fibrosis. The fibrosis may present as fibrosis of the lung, liver or heart. Administration of metal chelators to these patients can reduce the likelihood of worsening these conditions by exposure to gadolinium.

Similarly, aspects of the novel methods are designed to treat, reduce or ameliorate the effects of gadolinium exposure that render a patient at increased risk of developing a hypersensitivity condition. Examples of such hypersensitivity conditions include food allergies, a drug allergies and allograft rejection.

Other aspects of the inventive methods involve treating gadolinium toxicity in a patient comprising administering to the patient a pharmaceutically effective amount of hepcidin or a derivative thereof. The hepcidin or hepcidin derivative may be administered to the patient in oral, parenteral, or intraperitoneal form. Likewise, an aspect of the invention involves reducing the likelihood of a patient developing a condition induced by gadolinium toxicity comprising administering to the patient, prior to administering the gadolinium, hepcidin or a hepcidin derivative. Again, the hepcidin or hepcidin derivative may be administered in oral, parenteral, or intraperitoneal dosage form.

In embodiments of the present invention for preventing or reducing the risk of gadolinium toxicity, a metal chelator is administered in a single dosage about 1 to about 6 hours before administration of gadolinium to the patient, and after the gadolinium is administered to the patient, the metal chelator is administered to the patient as a series of follow-up dosages. The follow-up dosing regimen may comprise one dosage administered about every 12 hours for up to seven dosages, if the metal chelator is deferiprone, or may comprise one dosage administered about every 24 hours for up to four dosages, if the metal chelator is desferroxamine. Another embodiment of the present invention for treating gadolinium toxicity comprises administering to the patient a dosage of a pharmaceutically effective amount of deferiprone prior to gadolinium exposure, and thereafter administering a follow-up dosage of deferiprone about once every 12 hours for a total of about 96 hours. This dosing regimen may be suitable for patients with impaired renal function. For patients with normal renal function, the follow-up dosage of deferiprone may be reduced to once every 12 hours for a total of about 48 hours. In embodiments, the dosage comprises one immediate-release dosage form of deferiprone administered in combination with two extended release dosage forms of deferiprone. In a further embodiment, the immediate-release dosage form comprises about 900 mg of deferiprone, and each of the extended-release dosage forms comprises about 900 mg of deferiprone. In other embodiments, the follow-up dosage comprises the same immediate-release dosage form and extended-release dosage forms that are administered prior to the patient's exposure to gadolinium. In still further embodiments, the deferiprone dosage form may comprise a combination dosage form providing a ratio of one part immediate-release deferiprone to two parts extended-release deferiprone, so that the same dosage form may be administered throughout the dosage regimen.

In embodiments of the present invention for treating gadolinium toxicity, a metal chelator, for example desferroxamine, is administered to a patient in a cyclical pattern. In embodiments, such a cyclical pattern comprises administering the chelator to the patient for about 5 to about 7 days, and then withdrawing metal chelator from the patient for about 5 to about 7 days to complete one cyclical pattern. Thereafter, depending on the patient's iron status, renal status and other clinical measurements, the cyclical pattern may be repeated one or more times, until the patient's iron status shows that the patient is no longer at elevated risk of gadolinium toxicity.

### DETAILED DESCRIPTION OF THE INVENTION

In an embodiment of the invention, free gadolinium may be combined with an iron chelator and administered in combined form to a patient. For example, a combined gadolinium-iron chelator composition may be administered to a patient as a magnetic resonance contrast agent.

One embodiment of the invention includes diagnosing or otherwise identifying patients susceptible to gadolinium toxicity. Another embodiment includes identifying patients who are likely to develop gadolinium toxicity by measurement of an index of free iron levels, including but not limited to, catalytic iron, total serum iron-binding capacity, transferrin, ferritin, bone marrow iron stores, and hepatic iron stores (by histology or imaging) and correlating such measurements with increased risk for gadolinium toxicity. The increased risk for developing gadolinium toxicity corresponds with indices of iron status indicating the patient has greater levels of free iron than prior to exposure to gadolinium.

The term “correlating” refers to a measure of the relationship between two variables, for example, a patient's iron status and his risk for developing gadolinium toxicity. A correlation may be a quantitative measure, and optionally it may employ statistical analysis. Possible correlations can range, for example, from +1 to −1. A zero correlation on such a range indicates that there is no relationship between the variables. A perfect negative correlation of −1 indicates an inverse relationship, such that as one variable goes up, the other goes down. A perfect positive correlation of +1 indicates that both variables move in the same direction together. Less than perfect positive and negative correlations arise between 0 and +1 and 0 and −1, respectively. In the context of iron status, a positive correlation can be anticipated such that as indices of free iron levels increase, risk for gadolinium toxicity is expected to increase as well.

In certain embodiments of the invention, a patient is identified as having an increased risk of gadolinium toxicity by administering gadolinium to the patient; determining changes in the amounts of at least one index of the patient's iron status, such as serum iron, catalytic iron, total serum iron-binding capacity, transferrin, and ferritin; and identifying the patient as being at risk of gadolinium toxicity based on such change. In an embodiment of the invention, a change of the relevant index may be determined based on the measurement after administration of the gadolinium with respect to 1) a baseline value that is expected of a healthy individual, or 2) a baseline value of that particular patient. Alternatively, in certain other embodiments, a change value may be determined with respect to a measurement that is made prior to administration of gadolinium, for example, within minutes or approximately one to three hours prior to the gadolinium administration.

Yet another embodiment of the invention relates to a method of alleviating or otherwise reducing the symptoms of gadolinium toxicity by the administration of a therapeutically effective amount of a metal chelator. The metal chelator may, for example, be an iron chelator. The iron chelator may be a peptide comprising natural or non-natural amino acids (e.g., amino acids not found in nature), polyethylene glycol carbamates, lipophilic or nonlipophilic polyaminocarboxylic acids, polyanionic amines or substituted polyaza compounds, deferasirox, or deferitrin. In a preferred embodiment, the iron chelator is deferiprone (also known as the “L1”): (1,2-dimethyl-3-hydroxy-pyrid-4-one). In another preferred embodiment, the iron chelator is desferroxamine attached to hydroxyethyl starch. Other metal chelators, such as ethylenediamine tetra-acetic acid (EDTA), hydroxyquinolines (sometimes referred to as hydroxyquinolones) and N-acetylcysteine may be used for countering gadolinium toxicity as well. For example, hydroxyquinolines may be administered to a patient as a preventative measure to reduce the likelihood of developing symptoms of gadolinium toxicity, or as a therapeutic measure to alleviate the symptoms of such toxicity. Preferred hydroxyquinolines are 8-hydroxyquinolines and derivatives thereof, such as 5-[4-(2-hydroxethyl)piperazin-1-ylmethyl]-8-hydroxyquinoline.

As used in this specification, “free,” “free concentration” or “free form”, with respect to a metals, such as iron, copper, zinc, aluminum, or gadolinium, means the concentration of a metal that is available to participate in free radical reactions. “Free iron” and “catalytic iron” are terms that are interchangeably used to refer to iron that is available to participate in free radical reactions.

Free gadolinium is highly toxic and is therefore administered to humans as a chelate. For that reason, “gadolinium” as used in this specification means free gadolinium combined with a known gadolinium chelate in compound form. Known examples of gadolinium fall into one of two structurally distinct categories that are currently used: (a) the ‘macrocylic’ chelates such as Gd-DOTA (proHance), where Gd³⁺ is ‘caged’ in the pre-organized cavity of the ligand, and (b) the ‘linear’ chelates such as Gd-DTPA (Magnevist) or Gd-DTPA-BMA (Omniscan).

Several groups of patients may be susceptible to mobilization of metals, such as iron, aluminum, or zinc, which in turn may contribute to gadolinium toxicity, including NSF. For example, patients with chronic renal insufficiency may be particularly susceptible to tissue iron overload because of exogenous treatment with iron preparations, blood transfusions, hemolysis, hepatitis C infection, malnutrition resulting in low total iron-binding capacity, urinary protein losses of transferrin and, importantly, a possible direct effect of prolonged retention of gadolinium contrast. In addition, gadolinium, by inhibiting the reticulo-endothelial system and by its systemic inflammatory effect, could lead to low levels of iron-binding proteins such as transferrin, making the patient more susceptible to iron overload. Clinical data indicates that significant quantities of gadolinium can be detected in the skin, heart, aorta, kidney, lungs, liver, and spleen of patients suffering from NSF. Aspects of the invention, which are exemplified by—but not limited to—the embodiments, include the use of one or more metal chelators in connection with free gadolinium, or gadolinium-containing agents. In this aspect, the invention was devised to address the toxic effects thought to be associated with free gadolinium localized in mammalian tissues.

There are a number of clinically useful metal chelators in the practitioner's arsenal. Iron chelators are commercially available or can be synthesized or purified from biological sources using routine procedures. Exemplary descriptions and discussions of iron chelators known in the art may be found in references known to those of ordinary skill in the art, for example: U.S. Pat. No. 5,047,421; U.S. Pat. No. 5,424,057; U.S. Pat. No. 5,721,209 and U.S. Pat. No. 5,811,127. The chelation properties of hydroxyquinolines are discussed in U.S. Pat. No. 6,855,711 and U.S. Patent Publication No. 2006/0234927.

Pre-clinical studies investigating the safety of gadodiamide with both acute and sub-acute overdose in animals are known to those of skill in the art. With high doses (e.g., 1 mmol/kg), gadodiamide has caused significant hemodynamic effects with a lowering of systemic vascular resistance and blood pressure. Arrhythmias have been noted with high doses of gadopentetate dimeglumine injection. Several of these sub-acute toxic manifestations were thought to be related to zinc deficiency although serum zinc levels were found to be normal.

Reported clinical descriptions of gadolinium toxicity include increased incidence of acute renal failure (ARF) in CKD patients, NSF and sporadic case reports of acute pancreatitis, encephalopathy and hemolytic anemia. In addition, gadolinium appears to be associated with acute inflammatory responses, cardiac and cardiovascular fibrosis, and fibrotic complications of the skin.

Additional mechanisms contributing to iron-related gadolinium toxicity may include the effect of gadolinium on hepatic Kupffer cells. In normal health, iron overload and inflammation stimulates hepatic Kupffer cells to release interleukin-6, which in turn stimulates hepatocytes to synthesize and release hepcidin. Hepcidin, a 25-amino peptide, is a negative regulator of iron absorption in the gut and iron release from macrophages. Its effects on iron transport are mediated through inhibition of ferropontin.

Gadolinium may paradoxically decrease hepcidin synthesis and release, thus leading to a state of persistent iron overload contributing to gadolinium toxicity. Evidence indicates that gadolinium promotes the depletion of hepatic Kupffer cells, thereby impairing antigen specific tolerance. Patients experiencing inhibition of antigen specific tolerance have been found to be at increased risk of developing a hypersensitivity phenomenon such as food or drug allergies. Such patients are also more prone to allograft rejection than individuals not exposed to gadolinium. One embodiment of the invention relates to diagnosing an increased risk for developing gadolinium toxicity by measuring a predetermined decrease in hepcidin levels of a patient after administration of gadolinium to the patient. In another embodiment, gadolinium toxicity may be moderated, alleviated or treated by administration of hepcidin or any related compound that may increase the synthesis of hepcidin. Similarly, clinicians may utilize the invention to identify patients at risk for impaired antigen specific tolerance and hypersensitivity phenomenon. Thus, another embodiment of the invention is the identification of patients likely to develop drug allergies, food allergies or allograft rejection due to exposure to gadolinium. Administration of a metal chelator to these patients may reduce or even eliminate the anticipated impairment of antigen specific tolerance.

Gadolinium toxicity syndrome presents in some patients with symptoms of the development of renal dysfunction—such as acute kidney injury—nephrogenic systemic fibrosis, cardiovascular disease—such as atherosclerosis and vascular calcification associated with cardiovascular morbidity and mortality. Still other patients may manifest gadolinium toxicity as a disorder of accelerated senescence, such as osteoporosis, vascular calcification, skin wrinkling, dementia, basal cell and squamous cell skin cancers, and other cancers associated with senescence, and/or a progression of pre-existing bone density impairment. Individual patients may manifest only some of these findings. In other patients, gadolinium toxicity may manifest as a skin disorder, such as cutaneous lesions, progressive skin hardening, tethering, and hyperpigmentation. Additionally, patients could manifest with cutaneous inflammation or necrosis, anemia, thrombocytopenia, abnormal liver function tests, acute pancreatitis, hypogonadism, hemodynamic changes, arrhythmia, hypocalcemia, delayed hypercalcemia, and venous thromboembolism.

Patients at risk for gadolinium toxicity include patients with acute or chronic kidney disease who have underlying iron overload from diverse causes including chronic liver disease, hepatitis C, hemochromatosis, diabetes, iron overload due to transfusion or iron therapy, hemolytic anemia and reabsorbing hematomas. Additional patients at risk are those with low iron-binding capacity secondary to urinary losses of transferrin, chronic liver disease, sepsis, malnutrition and uremic inflammation. Moreover, patients receiving metal ions (for example, iron, aluminum, zinc, copper, lanthanum, gallium) or polycations (Vancomycin), either orally or parenterally, are also at risk for developing gadolinium toxicity.

A common thread of gadolinium toxicity syndrome appears to be an affinity demonstrated by gadolinium for areas of inflammation and/or fibrosis in mammalian systems. Gadolinium tends to localize in areas of pre-existing fibrosis, such as in the cardiac tissues of patients having cardiac damage. For example, patients with ventricular dysfunction may develop myocardial fibrosis. Fibrosis is also prevalent in cardiac tissue post-infarct. The localization of gadolinium in cardiac tissue, such as cardiac blood vessels, may result in decreased cardiac function, exacerbating a pre-existing heart condition, such as heart failure. Therefore, persons having a history of cardiac damage, cardiovascular disease or other inflammation-related damage to the circulatory system are at increased risk of developing gadolinium toxicity in comparison to persons having no history of cardiac or cardiovascular disease. These at-risk patients are more likely to experience increased levels of morbidity and mortality as a result of exposure to gadolinium. An embodiment of the invention includes the administration of a metal chelator to prevent, treat, or otherwise alleviate gadolinium-induced cardiovascular disease or cardiac damage.

Similarly, gadolinium may accumulate in the inflamed/fibrosed blood vessels and tissues of other organs, such as kidneys, thereby increasing the progression of kidney disease. Gadolinium appears to be attracted to areas of fibrosis, including areas within the kidneys. Gadolinium may also induce nephrogenic fibrosis due to localization at sites of inflammation within the kidney. An evaluation of patients suffering from nephrogenic systemic fibrosis demonstrated that gadolinium particles localized in the tissues of patients exposed to gadolinium-containing contrast agents. This appears to be due, at least in part, to iron mobilization in patients exposed to gadolinium (e.g., gadodiamide), which may lead to transmetallation and release of free gadolinium. Free gadolinium in combination with catalytic iron may synergistically coordinate, causing oxidative stress, inflammation, and tissue injury. Thus, gadolinium contrast administered to kidney patients for imaging purposes may place such patients at risk for increased progression of chronic kidney disease and acute renal failure due to gadolinium toxicity syndrome. These at-risk patients are more likely to experience increased levels of morbidity and mortality as a result of exposure to gadolinium than patients lacking peripheral inflammation. An embodiment of the invention includes the administration of a metal chelator to prevent, treat, or otherwise alleviate gadolinium-induced symptoms of acute kidney disease.

Patients afflicted with the autoimmune disease multiple sclerosis may also be at greater risk of developing gadolinium toxicity. Magnetic resonance imaging is often used to track the progression of multiple sclerosis by visualizing inflammatory multiple sclerosis lesions. Gadolinium chelates are typically used to show permeability of the blood brain barrier in these inflammatory multiple sclerosis lesions. Because gadolinium localizes in these inflammatory lesions of the central nervous system, multiple sclerosis patients exposed to gadolinium may experience iron-mediated transmetallation reactions in these lesions. Such multiple sclerosis patients are more likely to experience progression of their neurodegenerative symptoms—and thus a higher degree of morbidity—than patients who do not have iron-mediated transmetallation reactions in their brain lesions. Such patients can be expected to have an accelerated rate of mortality. An embodiment of the invention includes the administration of a metal chelator to prevent, treat, or otherwise alleviate gadolinium-induced exacerbation of multiple sclerosis.

Metal accumulation in tissue appears to induce symptoms of accelerated senescence, such as osteoporosis, vascular calcification, skin wrinkling, dementia, and cancer. Evidence indicates this may be due to gadolinium-induced inhibition of transient receptor potential channel V5 (TRPV5). TRPV5 is stimulated by klotho, an anti-aging hormone generated by the kidney. Gadolinium may promote aging via inhibition/antagonism of TRPV5 function, or by interfering with the production or function of klotho, a TRPV5 agonist. Likewise, the negative effects of gadolinium on bone deposition may induce or exacerbate the loss of bone density in patients. Thus, patients undergoing magnetic resonance imaging to track the progression of, for example, osteoporosis may experience an increased risk of bone fractures due to exposure to gadolinium-containing contrast agents. Patients at risk for gadolinium toxicity may be identified by measuring iron status in bone marrow. Iron status may be identified by histopathology, analytical methods (such as inductively-coupled mass spectrometry) or by radiologic methods (such as magnetic resonance imaging). An embodiment of the invention includes the administration of a metal chelator to prevent, treat, or otherwise alleviate gadolinium-induced symptoms of osteoporosis and other manifestations of accelerated senescence.

Patients at risk of developing gadolinium toxicity may be treated with a pharmaceutically effective amount of a metal chelator to reduce the physiological effects of free gadolinium exposure. A pharmaceutically or therapeutically effective amount of such a metal chelator will complex with free metal cations in an amount sufficient to decrease oxidative stress and attendant tissue damage in a mammalian system. For example, patients may receive an iron chelator before, concurrently with, or after injection of a gadolinium contrast agent. The iron chelator may be, for example, desferroxamine, deferiprone, and deferasirox, either alone or in combination.

Metal chelators may be administered to persons exposed to gadolinium as a preventative measure to reduce the likelihood of developing symptoms of gadolinium toxicity, or as a therapeutic measure to alleviate the symptoms of such toxicity. In embodiments of a method of the present invention, a metal chelator is administered one to six hours prior to administration of a gadolinium contrast agent, and then at intervals of about 12 hours for a total of about seven administrations following the administration of the gadolinium contrast agent. In other embodiments for prevention or reduction of the risk of gadolinium toxicity following administration of a gadolinium contrast agent, desferroxamine may be administered subcutaneously at a dosage of 5-50 mg/Kg/day, for about 5 to about 7 days per week, more particularly at a dosage 25 mg/Kg/day, in single or divided doses. Likewise, for this indication, deferiprone may be administered orally at a dosage of 10-100 mg/Kg/day, more particularly at a dosage of 50-75 mg/Kg/day, in single or divided doses, until tests confirm that the risk of gadolinium toxicity has been reduced to an acceptable level. Similarly for this indication, deferasirox (e.g., Exjade®, Novartis Pharmaceutical Corporation) may be administered orally at a dosage of 3-30 mg/Kg/day, more particularly at a dosage of 15 mg/Kg/day, in single or divided doses, again until tests confirm that the risk of gadolinium toxicity has been reduced to an acceptable level.

Other metal chelators, such as EDTA, hydroxyquinolines and N-acetylcysteine may be used for countering gadolinium toxicity as well. For example, hydroxyquinolines may be administered to a patient at a dosage range of about 10 mg/Kg to about 100 mg/Kg per day, in single or divided doses, as a preventative measure to reduce the likelihood of developing symptoms of gadolinium toxicity, or as a therapeutic measure to alleviate the symptoms of such toxicity. Preferred hydroxyquinolines are 8-hydroxyquinolines and derivatives thereof, such as 5-[4-(2-hydroxethyl)piperazin-1-ylmethyl]-8-hydroxyquinoline.

Modes of administration of the metal chelators and other agents encompassed by the present invention for reducing the risk of, preventing, ameliorating, or treating gadolinium toxicity include oral, subcutaneous, intravenous, parenteral or other modes of administration appropriate in light of the agent to be administered and the condition of the patient, as apparent in light of this specification.

The invention is further illustrated by the following non-limiting examples.

### EXAMPLE 1

A 60-year old male with chronic kidney disease received gadolinium contrast for an angiogram of his renal arteries. Upon exposure to gadolinium contrast, he developed iron mobilization (serum ferritin increased from 250 ng/dL to 6000 ng/dL), increased C-reactive protein, acute kidney injury needing hemodialysis, anemia, thrombocytopenia, hypotension and nephrogenic systemic fibrosis (NSF). He died 8 weeks later from the illness. This patient could have been treated according to one or more embodiments of the invention to reduce the likelihood of his developing symptoms of gadolinium toxicity, such as NSF, thereby reducing his morbidity and the likelihood of mortality. In particular, according to an embodiment of the invention, a treatment protocol for this patient would have included administering pharmaceutically effective doses of an iron chelator beginning 24-48 hours before administering gadolinium contrast. Specifically, as evaluated according to an embodiment of the invention, this patient would have received orally administered deferiprone at a dosage of 50 mg/Kg/day before gadolinium treatment was initiated. The dosage regimen—adjusted as needed in view of serum ferritin levels over time and renal function, and continued for a few weeks after gadolinium contrast exposure—is expected to have improved this patient's medical condition.

### EXAMPLE 2

A 50-year old male presenting with chronic kidney disease and pituitary adenoma undergoes gamma knife resection of adenoma and at that time is exposed to gadolinium contrast. He presents 1 year later with progressive kidney disease, severe bone pain and osteoporosis. The patient demonstrates clinical signs of having aged by at least 10-15 years within the last year. If, however, the patient had been evaluated according to an embodiment of the invention, he would have been administered an iron chelator before and after gadolinium exposure. It is believed that this patient's symptoms of gadolinium toxicity—accelerated senscence, progressive kidney disease and osteoporosis—would have been ameliorated or even prevented. Specifically, as per an embodiment of the invention, the patient at least would have received, before gadolinium treatment was initiated, deferiprone at an oral dosage of 25 mg/Kg, administered one to three times daily before gadolinium treatment was initiated. Depending on, and responsive to, the patient's symptoms and renal function, the deferiprone dosing would have continued for several weeks after gadolinium exposure as well. The patient's deferiprone dosage would have been adjusted as needed in view of measures of his iron status before and after gadolinium exposure.

### EXAMPLE 3

A 55-year old female was diagnosed with chronic kidney disease and mild congestive heart failure in 2005. She had a skin biopsy in 2005 that showed dermal fibrosis. In March 2006, she was administered gadolinium contrast for magnetic resonance imaging of her brain. Over the next 3 months, this patient's congestive heart failure worsened, necessitating ventilatory support. The patient succumbed to her symptoms and died. In her case, medical evidence evaluated according to an embodiment of the invention suggests that gadolinium contrast was attracted to pre-existing myocardial fibrosis, resulting in gadolinium deposition in her heart. This predicted deposition of gadolinium would be expected to further worsen the patient's heart failure, eventually resulting in her death. Administering an iron chelator before and after exposure to gadolinium contrast, based on the retrospective review of her history according to an embodiment of the invention, is predicted to have prevented gadolinium transmetallation in her heart. In particular, following the embodiment of the invention, the patient should have been administered deferiprone at an oral dosage of 50 mg/Kg/day, in single or divided doses, for one to three days before gadolinium treatment was initiated. The deferiprone dosing would have continued for several weeks after gadolinium exposure. The patient's deferiprone dosage would have been adjusted as needed in view of measures of her iron status before and after gadolinium exposure and her renal function. Therefore, her morbidity probably would have been reduced substantially, and quite possibly her death at this time would have been avoided.

### EXAMPLE 4

A 45-year old male with hepatitis C, type 2 diabetes mellitus and normal renal function was admitted to the hospital with cellulitis of his left leg. He underwent magnetic resonance imaging with gadolinium contrast to evaluate for osteomyelitis. The patient was administered naficillin for treatment of his cellulitis. A week later, he developed allergic acute interstitial nephritis with eosinophilia accompanied by a pruritic skin rash. In his case, according to an embodiment of the invention, administering an iron chelator before administering gadolinium contrast is predicted to have prevented gadolinium transmetallation in the liver, hepatic Kupffer cell depletion, and his subsequent drug-specific antigen tolerance. In particular, according to an embodiment of the invention, in light of the patient's symptoms, the patient would have been administered deferiprone at an oral dosage of 25 mg/Kg, administered one to three times daily, before gadolinium treatment was initiated. The deferiprone treatment would have continued for several weeks after gadolinium exposure. During this period, the patient's status would have been monitored, and his deferiprone dosage would have been adjusted as needed in view of measures of his iron status before and after gadolinium exposure. Following this regimen, this patient's morbidity probably would have been reduced substantially, as his allergic reactions could have been greatly diminished, or avoided altogether.

### EXAMPLE 5

An immediate-release deferiprone tablet may be formulated according to the following ingredients and proportions, using techniques apparent to one of skill in the tablet formulation art in light of this specification:

| | Ingredient | Amount |
|---|---|---|
| 1 | Deferiprone | 900 mg |
| 2 | Pre-gelatinized starch | 47 mg |
| 3 | Povidone K 29/32 | 28 mg |
| 4 | Purified Water | Qs |
| 5 | RoCoat (Riboflavin 33.33%) | 10 mg |
| 6 | Crospovidone | 10 mg |
| 7 | Magnesium Stearate | 10 mg |
| | Total Weight | 1005 mg |

### EXAMPLE 6

An immediate-release deferiprone tablet may be formulated according to the following ingredients and proportions, using techniques apparent to one of skill in the tablet formulation art in light of this specification:

| | Ingredient | Amount |
|---|---|---|
| 1 | Deferiprone | 900 mg |
| 2 | Pre-gelatinized starch | 47 mg |
| 3 | Povidone K 29/32 | 28 mg |
| 4 | Purified Water | Qs |
| 5 | Crospovidone | 10 mg |
| 6 | Magnesium Stearate | 10 mg |
| 7 | Opadry Film-Coat | 20 mg |
| | Total Weight | 1015 mg |

### EXAMPLE 7

An extended-release deferiprone tablet may be formulated according to the following ingredients and proportions, using techniques apparent to one of skill in the tablet formulation art in light of this specification:

| | Ingredient | Amount |
|---|---|---|
| 1 | Deferiprone | 900 mg |
| 2 | Hydroxypropyl MethylCellulose K 100M | 50 mg |
| 3 | Povidone K 29/32 | 15 mg |
| 4 | Purified Water | Qs |
| 5 | RoCoat (Riboflavin 33.33%) | 10 mg |
| 6 | Magnesium Stearate | 10 mg |
| | Total Weight | 985 mg |

### EXAMPLE 8

An extended-release deferiprone tablet may be formulated according to the following ingredients and proportions, using techniques apparent to one of skill in the tablet formulation art in light of this specification:

| | Ingredient | Amount |
|---|---|---|
| 1 | Deferiprone | 900 mg |
| 2 | Hydroxypropyl MethylCellulose K 100M | 50 mg |
| 3 | Povidone K 29/32 | 15 mg |
| 4 | Purified Water | Qs |
| 5 | Magnesium Stearate | 10 mg |
| 6 | Opadry Film-Coat | 20 mg |
| | Total Weight | 995 mg |

It is appreciated that certain features of the invention, which are, for clarity, described in the text of separate examples and embodiments, may also be provided in combination in a single embodiment. Likewise, various features of the invention described in the context of a single embodiment, may also be provided separately or in any suitable sub-combination, as will be evident to those having skill in the art.

While the invention is exemplified herein by specific embodiments thereof, many alternatives, modifications and variations of those embodiments will be apparent to those skilled in the art. Accordingly, all such alternatives, modifications and variations that fall within the spirit and broad scope of the appended claims are embraced as embodiments of the invention. Citation or identification of references herein shall not be construed as an admission that such references constitute available as prior art to the claims.

What is claimed is:

1. A method of identifying a patient at elevated risk for gadolinium toxicity comprising
determining a clinical condition of the patient associated with or predisposing the patient to fibrosis, inflammation, or both; and
correlating the clinical condition with elevated risk for gadolinium toxicity.

2. The method of claim 1, wherein the clinical condition is selected from the group consisting of nephrogenic systemic fibrosis, acute kidney injury, cardiovascular disease and accelerated senescence.

3. The method of claim 1, wherein the clinical condition is accelerated senescence manifesting as one or more of vascular calcification, dementia, osteoporosis, skin wrinkling, basal cell skin cancer or squamous cell skin cancer.

4. A method of identifying a patient at elevated risk for gadolinium toxicity comprising:
measuring an index of patient iron status selected from the group consisting of serum total iron-binding capacity of the patient, transferrin level of the patient, transferrin saturation of the patient, concentration of tissue iron in the liver of the patient and concentration of tissue iron in bone marrow of the patient; and
correlating a value of the index with elevated patient risk for gadolinium toxicity.

5. The method of claim 4 wherein the index is serum total iron-binding capacity and the value of the index is less than about 200 μg/dL.

6. The method of claim 4 wherein the measuring and correlating are carried out prior to administration of gadolinium.

7. A method of identifying a patient at increased risk for gadolinium toxicity comprising:
administering gadolinium to the patient;
measuring an increase in transferrin saturation of the patient; and
correlating a an increase in transferrin saturation more than about 25% of the patient with increased risk of the patient for gadolinium toxicity.

8. The method of claim 7, wherein the increase in transferrin saturation of the patient is determined relative to one of: a baseline value of transferrin saturation expected of a healthy patient, a baseline value of the patient, and a value of transferrin saturation for the patient measured prior to the administering.

9. A method of identifying a patient at increased risk for gadolinium toxicity comprising:
administering gadolinium to the patient;
measuring serum iron of the patient; and
correlating an increase of more than about 50% in serum iron of the patient with increased risk of the patient for gadolinium toxicity.

10. A method of identifying a patient at increased risk for gadolinium toxicity comprising:
administering gadolinium to the patient;
measuring serum ferritin of the patient; and
correlating an increase of more than 50% in serum ferritin or a serum ferritin value of greater than about 500 ng/mL, with increased risk of the patient for gadolinium toxicity.

11. A method of identifying a patient at increased risk for gadolinium toxicity comprising:
administering gadolinium to the patient;
measuring oxidative stress of the patient; and
correlating an increase of more than about 50% in oxidative stress with increased risk of the patient for gadolinium toxicity.

12. A method of identifying a patient increased at risk for gadolinium toxicity comprising:
administering gadolinium to the patient;
measuring catalytic iron of the patient; and
correlating an increase of more than about 50% in catalytic iron with an increased risk of the patient for gadolinium toxicity.

13. A method of identifying a patient at increased risk for gadolinium toxicity comprising:
administering gadolinium to the patient;
measuring the hepcidin level of the patient; and
correlating a decrease of more than about 20% in the hepcidin level with an increased risk of the patient for gadolinium toxicity.

14. A method for reducing the likelihood of developing gadolinium toxicity in a human exposed to gadolinium comprising administering an amount of a metal chelator to the human effective to reduce the likelihood of the human developing conditions induced by gadolinium toxicity.

15. The method of claim 14, wherein gadolinium toxicity is manifested as one or more conditions selected from the group consisting of nephrogenic systemic fibrosis, acute kidney injury, cardiovascular disease and accelerated senescence.

16. The method of claim 15, wherein accelerated senescence manifests one or more of vascular calcification, osteoporosis, dementia, skin wrinkling, squamous cell carcinoma or basal cell carcinoma.

17. The method of claim 14, wherein the metal chelator is administered in combination with gadolinium.

18. The method of claim 14, wherein the metal chelator chelates iron.

19. The method of claim 14, wherein the metal chelator is selected from the group consisting of ethylenediamine tetra-acetic acid, N-acetylcysteine, hydroxyquinoline, deferiprone, deferasirox, deferitrin, deferoxamine, polyanionic amines, substituted polyaza compounds, 2-pyridylcarboxyaldehyde isonicotinoyl hydrazones, di-2-pyridylketone isonicotinoyl hydrazones, di-2-pyridylketone thiosemicarbazones, and 3-aminopyridine-2-carboxaldehyde-thiosemicarbazone.

20. The method of claim 14, wherein the metal chelator is administered orally, subcutaneously, intravenously or intraperitoneally.

21. The method of claim 19, wherein the metal chelator comprises deferiprone administered orally at a dosage from about 10 mg/Kg/day to about 100 mg/Kg/day.

22. The method of claim 19, wherein the metal chelator comprises desferroxamine administered subcutaneously at a dosage from about 5 mg/Kg/day to about 50 mg/Kg/day for five consecutive days to seven consecutive days.

23. The method of claim 19, wherein the metal chelator comprises desferasirox administered orally at a dosage from about 3 mg/Kg/day to about 30 mg/Kg/day.

24. The method of claim 14, wherein the patient has a pre-existing condition that can be exacerbated by gadolinium exposure.

25. The method of claim 24, wherein the pre-existing condition is selected from the group consisting of multiple sclerosis, chronic kidney disease, congestive heart failure, inflammation and fibrosis.

26. The method of claim 25, wherein the fibrosis comprises fibrosis of the lung, liver or heart.

27. The method of claim 14, wherein the patient is at increased risk of developing a hypersensitivity condition selected from the group consisting of a food allergy, a drug allergy, and allograft rejection.

28. A method of treating gadolinium toxicity in a patient exposed to gadolinium comprising administering to the patient a pharmaceutically effective amount of a metal chelator.

29. The method of claim 28, wherein the metal chelator is an iron chelator.

30. The method of claim 29, wherein the iron chelator chelates free iron.

31. The method of claim 28, wherein the metal chelator is selected from the group consisting of ethylenediamine tetra-acetic acid, N-acetylcysteine, 8-hydroxyquinolines, deferiprone, deferasirox, deferitrin, deferoxamine, polyanionic amines, substituted polyaza compounds, 2-pyridylcarboxyaldehyde isonicotinoyl hydrazones, di-2-pyridylketone isonicotinoyl hydrazones, di-2-pyridylketone thiosemicarbazones, and 3-aminopyridine-2-carboxaldehyde-thiosemicarbazone.

32. The method of claim 28, wherein the metal chelator is administered orally, subcutaneously, intravenously or intraperitoneally.

33. The method of claim 31, wherein the metal chelator comprises deferiprone administered orally at a dosage from about 10 mg/Kg/day to about 100 mg/Kg/day.

34. The method of claim 31, wherein the metal chelator comprises desferroxamine administered subcutaneously at a dosage from about 5 mg/Kg/day to about 50 mg/Kg/day for 5 consecutive days to 7 consecutive days.

35. The method of claim 31, wherein the metal chelator comprises desferasirox administered orally at a dosage from about 3 mg/Kg/day to about 30 mg/Kg/day.

36. The method of claim 32, wherein the metal chelator is deferiprone administered in a single dosage about 1 to about 6 hours before administration of gadolinium to the patient, and thereafter the deferiprone is administered to the patient post-gadolinium administration as a series of follow-up dosages comprising one dosage administered about every 12 hours for up to seven dosages.

37. The method of claim 32, wherein the metal chelator is desferroxamine administered in a single dosage about 1 to about 6 hours before administration of gadolinium to the patient, and thereafter the desferroxamine is administered to the patient post-gadolinium administration as a series of follow-up dosages comprising one dosage administered about every 24 hours for up to four dosages.

38. The method of claim 34, wherein the desferroxamine is administered in a cyclical pattern, comprising administering the desferroxamine to the patient for about 5 to about 7 days, and then withdrawing desferroxamine from the patient for about 5 to about 7 days to complete one cyclical pattern of desferroxamine treatment, and thereafter administering one or more cyclical patterns of desferroxamine until one or more indices of patient iron status fails to correlate with an elevated patient risk for gadolinium toxicity.

39. The method of claim 28, wherein the patient has a pre-existing condition that can be exacerbated by gadolinium exposure.

40. The method of claim 39, wherein the pre-existing condition is selected from the group consisting of multiple sclerosis, chronic kidney disease, congestive heart failure, inflammation and systemic fibrosis.

41. The method of claim 40, wherein the fibrosis comprises lung fibrosis, liver fibrosis, or cardiac fibrosis.

42. The method of claim 28, wherein the patient is at increased risk of developing a gadolinium-induced hypersensitivity condition selected from the group consisting of a food allergy, a drug allergy, and allograft rejection.

43. The method of claim 28, wherein the patient is at increased risk of developing a gadolinium-induced condition selected from the group consisting of nephrogenic systemic fibrosis, acute kidney injury, cardiovascular disease and accelerated senescence.

44. The method of claim 43, wherein the accelerated senescence manifests as one or more conditions selected from the group consisting of vascular calcification, dementia, osteoporosis, skin wrinkling, basal cell skin cancer and squamous cell skin cancer.

45. A method for treating gadolinium toxicity in a patient comprising administering to the patient a pharmaceutically effective amount of hepcidin or a derivative thereof, the hepcidin or hepcidin derivative being administered to the patient in oral, parenteral, or intraperitoneal form.

46. A method of reducing the likelihood of a patient developing a condition induced by gadolinium toxicity comprising administering to the patient, prior to administering the gadolinium, hepcidin or a hepcidin derivative in oral, parenteral, or intraperitoneal dosage form.

47. A method of administering gadolinium to a patient comprising administering a composition comprising free gadolinium in combination with an iron chelator.

48. A method of treating gadolinium toxicity in a patient exposed to gadolinium comprising administering to the patient a dosage of a pharmaceutically effective amount of deferiprone prior to gadolinium exposure, and thereafter administering a follow-up dosage of deferiprone about once every 12 hours for a total of about 96 hours.

49. The method of claim 48, wherein the dosage comprises one immediate-release dosage form of deferiprone administered in combination with two extended release dosage forms of deferiprone.

50. The method of claim 49, wherein the immediate-release dosage form comprises about 900 mg of deferiprone, and each of the extended-release dosage forms comprises about 900 mg of deferiprone.

51. The method of claim 48, wherein the dosage comprises at least one dosage form comprising a ratio of one part immediate-release of deferiprone to two parts extended-release of deferiprone.
